(19) United States
(12) Patent Application Publication
(10) Pub. No.: US 2014/0273223 A1
(43) Pub. Date: Sep. 18, 2014

(54) MICRO-DEVICE FOR CULTURING CELLS, METHOD FOR MANUFACTURING SAME, AND METHOD FOR CULTURING CELLS USING THE MICRO-DEVICE FOR CULTURING CELLS

(71) Applicants: Yoon-Kyoung Cho, Ulju-gun (KR); Hyundoo Hwang, Ulju-gun (KR)

(72) Inventors: Yoon-Kyoung Cho, Ulju-gun (KR); Hyundoo Hwang, Ulju-gun (KR)

(73) Assignee: UNIST ACADEMY-INDUSTRY RESEARCH CORPORATION, Ulju-gun (KR)

(21) Appl. No.: 14/233,129

(22) PCT Filed: Sep. 29, 2011
(86) PCT No.: PCT/KR2011/007192

§ 371 Date: Mar. 5, 2014

(30) Foreign Application Priority Data
Jul. 15, 2011 (KR) 10-2011-0070232

(51) Int. Cl. C12M 3/06; C12N 5/00; C12M 1/12

(52) U.S. Cl. CPC C12M 23/16; C12M 25/14; C12N 5/0062
USPC 435/396; 435/289.1; 435/299.1; 264/328.1; 264/494; 264/293; 264/255

(57) ABSTRACT

Disclosed is micro-device for culturing cells comprising: a plurality of fluid paths through which fluid moves; and at least one inlet port for injecting fluid to the fluid paths, said fluid paths communicating with each other and being different in height from each other. In the cell culture device having a plurality of fluid paths, cells can be cultured by introducing a polymeric material to at least one fluid path having a relatively low height; solidifying the polymeric material to form a 3-dimensional scaffold; and injecting fluid for cell culture to a fluid path in contact with the 3-dimensional scaffold.

### TECHNICAL FIELD

The present invention relates to a micro-device for culturing cells, and a method for fabricating the same. More particularly, the present invention relates to a micro-device for culturing cells which allows cells to grow at low cost and a high efficiency, a method for fabricating the same, and a method for culturing cells using the same.

### BACKGROUND ART

Classical cell-culturing methods using culture dishes consume much culture media, giving rise to a significant increase in cost. In addition, such a large dimension of classical cell cultures reduces work efficiency in multi-step processes which should be manually operated with care.

When applied to three-dimensional cultures, classical cell-culturing methods, which are typical of two-dimensional cultures, require a large amount of expensive polymeric materials for use as scaffolds.

To overcome the limitations of classical cell culturing methods, microfluidic cell culture systems have recently been developed to grow cells in micro-channels with a dimension of tens to hundreds of micrometers, such as a microfluidic 3-dimensional cell culture system disclosed in Korean Patent No. 10-0733914 (issued on Jun. 25, 2007).

Another example may be met in Korean Patent Publication No. 2011-0003526 (issued on Jan. 12, 2011) which discloses a three-dimensional microfluidic platform comprising a three-dimensional scaffold, constructed within a microfluidic channel in dimensions of tens to hundreds micrometers, in which liquid flows through opposite lateral paths of the scaffold, whereby cells can be cultured within the three-dimensional scaffold. The microfluidic channel establishes a simulated body condition with plural reagents therein under which the cells can grow well. Thus, this platform makes it possible to culture and analyze cells even with very small amounts of reagents, culture media, and cells, compared to classical cell-culturing methods, demonstrating its useful applications in high sensitivity cell analysis, the assay of multi-drug interactions, and the cultivation of cells for the simulation of in vivo vessels and various diseases such as artherosclerosis, cerebral infarction, etc., and thus as a basic platform for the development of new drugs.

Figured to accurately control microfluids in cooperation of plural pumps, conventional microfluidic cell culture systems are, however, need expensive facilities, complicated controllers, elaborate manual operation, and multi-step processes.

When microstructures are used to form a three-dimensional scaffold using surface tension in conventional microfluidic devices, the stability of the three-dimensional scaffold is very closely correlated with the surface tension, injection pressure and temperature of the liquid, and the size, shape, number and interval of the microstructures. Hence, conventional micro-device for culturing cells is not readily employed.

Moreover, when the type of the 3-dimensional scaffold is changed according to the kind of the cells to be cultured, full account must be taken of not only the size, morphology, number and intervals of the microstructures, but also the surface properties of the materials of the microstructures. Also, a series of processes is needed to maintain the requirements for the microstructures.

In addition, the microfluidic architecture in which a space between the 3-dimensional scaffold and the fluid is partially blocked by the microstructure is apt to change in the fluid flow through the 3-dimensional scaffold, and the concentration gradients of the media and chemicals provided to cells, depending on the morphology, size, number and interval of the post. For this reason, it is impossible to culture cells under the condition of constant fluid flows and concentration gradients. This uncertainty makes it difficult to accurately control the minute environment in the microfluidic cell culture system, which leads to erroneous data of biological experiments and analysis.

A micro-process is requisite for fabricating such conventional micro-device for culturing cells. Particularly, the microstructures with a size of tens of micrometers are constructed using a micro-etching process, such as photolithography or reactive ion etching, which consumes much time and cost.

If occurring in this process, an error, although minute, has a significant influence on the size and shape of the post, which results in failing in the fabrication of the 3-dimensional scaffold. Thus, the success rate of fabricating the cell culture device is not high, incurring additional expense and time loss.

### DISCLOSURE

### Technical Problem

Accordingly, the object of the present invention is to provide a micro-device for culturing cells which allows cells to grow at low cost and a high efficiency, and a method for fabricating the same.

### Technical Solution

In accordance with an aspect thereof, the present invention provides a micro-device for culturing cells, comprising: a plurality of fluid paths through which fluid moves; and at least one inlet port for injecting fluid to the fluid paths, said fluid paths communicating with each other and being different in height from each other.

In accordance with another aspect thereof, the present invention provides a method for manufacturing micro-device for culturing cells, comprising: forming a first microstructure having the same height as a first fluid path on an area of a flat plate, said area corresponding to a sum of the first fluid path and a second fluid path lower in height than the first fluid path; forming a second microstructure having a height corresponding to a difference between the heights of the first fluid path and the second fluid path on the first microstructure; applying a liquid material for forming the fluid paths to the first microstructure and the second microstructure, followed by solidifying the liquid material; overlying the solidified material to a substrate to form the first fluid path and the second fluid path, with a connection therebetween; introducing a polymeric material to the second fluid path to form a 3-dimensional scaffold; and injecting fluid to the first fluid path.

In accordance with a further aspect thereof, the present invention provides a method for culturing cells using the micro-device for culturing cells having a plurality of fluid paths, comprising: introducing a polymeric material to at least one fluid path having a relatively low height; solidifying the polymeric material to form a 3-dimensional scaffold; and injecting fluid for cell culture to a fluid path in contact with the 3-dimensional scaffold.

### Advantageous Effects

As described hitherto, the micro-device for culturing cells of the present invention can be fabricated at low cost using a simple process, and allows cells to grow at a specific site therein even without an elaborate device such as a pump. Particularly, a 3-dimensional scaffold for use in cell culture, migration and differentiation can be readily established in a certain area of the microfluidic channel, so that the migration and differentiation of cells according to the flow of interstitial fluid or the concentration gradient of molecules can be readily and conveniently observed. Free of micro-posts which are used to stabilize 3-dimensional scaffolds of conventional devices, the micro-device for culturing cells of the present invention can be generally applied to various cells and scaffold materials. Hence, the device of the present invention performs biological experiments at low cost. All sides of the 3-dimensional scaffold are open to the fluid paths constructed in the device, so that the flow of interstitial fluid or the concentration gradient of molecules across the 3-dimensional scaffold can be elaborately controlled, enabling a precise experiment for cell migration and differentiation.

### DESCRIPTION OF DRAWINGS

FIG. 1 is a perspective view of a micro-device for culturing cells according to a preferred embodiment of the present invention.

FIG. 2 is a cross sectional view of the micro-device for culturing cells of FIG. 1.

FIG. 3 is a perspective view of a micro-device for culturing cells according to another embodiment of the present invention in which a 3-dimensional scaffold is established, with fluid flowing therein.

FIG. 4 is a cross sectional view of the micro-device for culturing cells of FIG. 3.

FIG. 5 is a cross sectional view of the micro-device for culturing cells according to one embodiment of the present invention depicting the growth of cells within the 3-dimensional scaffold.

FIGS. 6A and 6B are microscopic images of cells growing within the micro-device for culturing cells of FIG. 5.

FIG. 7 is a cross sectional view of the micro-device for culturing cells of the present invention, depicting the generation of interstitial flow across the 3-dimensional scaffold.

FIG. 8A is a perspective view illustrating an embodiment in which a concentration gradient of a compound of interest is established across the 3-dimensional scaffold by introducing a solution containing the chemical to one of the fluid paths and a solution free of the chemical to the other.

FIG. 8B is a graph depicting concentration gradients of a chemical of interest established across the 3-dimensional scaffold when the chemical is introduced to only one of the fluid paths.

FIG. 9 shows photographic images of cell migration within the 3-dimensional scaffold according to the concentration gradient.

FIG. 10 is a cross sectional view depicting a micro-device for culturing cells according to another embodiment of the present invention in which heterogeneous cells are cultured simultaneously.

FIG. 11 is a photographic image depicting the co-culturing of heterogeneous cells.

FIG. 12 is a cross sectional view depicting a micro-device for culturing cells according to another embodiment of the present invention in which heterogeneous cells are cultured simultaneously.

FIG. 13 is a photographic image illustrating an embodiment in which heterogeneous cells are co-cultured using the device of FIG. 12.

FIG. 14 is a cross sectional view illustrating an embodiment in which fluid is introduced to a relatively high-height fluid path.

FIG. 15 is a process flow depicting the construction of the microfluidic cell culture according to the present invention.

FIG. 16 is a schematic view showing microstructures fabricated by clipping tape fragments and overlaying them.

### BEST MODE

Reference should now be made to the drawings, in which the same reference numerals are used throughout the different drawings to designate the same or similar components.

With reference to FIG. 1, there is a perspective view of a micro-device for culturing cells according to one embodiment illustrative of the present invention, with a cross sectional view thereof provided in FIG. 2.

The micro-device for culturing cells of the present invention, as shown, comprises a first fluid path 200, having a predetermined height, through which fluids move; a second fluid path 201, having a lower height than the first fluid path 200, with a connection therebetween; a first inlet port 300 for injecting fluid into the first fluid path 200; and a second inlet port 301 for injecting fluid into the second fluid path 201.

A plate 100 for forming the first fluid path 200 and the second fluid path 201 is laid on a substrate 101 to form a composite. In this composite, each of the fluid paths 200 and 201, which are different in height from each other, may have one or more inlet ports 300 or 301. The fluid paths 200 and 201, having different heights to each other, with a connection therebetween, are located on the flat plane of the substrate 101.

Both the plate 100 for forming the fluid paths 200 and 201 and the substrate 101 are preferably transparent. In the present invention, they may be made of a plastic or glass selected from the group consisting of poly(dimethylsiloxane) (PDMS), polymethylmethacrylate (PMMA), polyacrylates, polycarbonates, polycyclic olefins, polyimides, polyurethanes, and a combination thereof.

More hydrophobic surfaces of both of the plate 100 for forming the fluid paths 200 and 201 are more advantageous for the flow of fluid. When the contact angle of the fluid to be injected is less than 90°, it can spontaneously proceed to the fluid paths 200 and 201 without the aid of a peripheral device. In contrast, when either of the plate 100 for forming the fluid paths 200 and 201, and the substrate 101 is hydrophobic or when the contact angle of the liquid to be injected is over 90° C., a peripheral device, such as a pump, for injecting fluid is needed.

Given a connection between the fluid paths 200 and 201 which have different respective heights, the flowing of fluid from the low-height path to the high-height path is restrained by the surface tension of the fluid injected whereas the fluid smoothly moves from the high-height path to the low-height path. Accordingly, the low-height path can be first filled with fluid by injecting fluid to the low-height path.

FIG. 3 is a perspective view of a micro-device for culturing cells according to another embodiment of the present invention in which a 3-dimensional scaffold is established, with fluid flowing therein. FIG. 4 is a cross sectional view of the micro-device for culturing cells of FIG. 3 in which a 3-dimensional scaffold is established, with fluid flowing therein.

The micro-device for culturing cells according to another embodiment of the present invention comprises a first fluid path 200 through which fluid 400 moves; a second fluid path 201 in contact with the first fluid path 200, having a lower height than the first fluid path 200; a three-dimensional scaffold 401; and one or more inlet ports 300 and 301 for injecting fluid into the paths.

A three-dimensional scaffold 401 is established in the second fluid path 201 having a lower height than the first fluid path 200. To this end, a material for the three-dimensional scaffold 401 is preferably introduced through the second inlet port 301 apart at the same distance from the substrate 101 as the height of the second fluid path 201. In this regard, the material for the three-dimensional scaffold 401 fills only the second fluid path 201.

Subsequently, the three-dimensional scaffold 401 is formed. The formation of the three-dimensional scaffold 401 may be accomplished by, for example, maintaining a temperature at which sol-gel polymerization may occur for a predetermined time, irradiating light of certain wavelength which can initiate polymerization, or adding a polymerization initiator. The factors which elicit the formation of the 3-dimensional scaffold 401, including temperatures, time, light wavelengths, and initiators, vary depending on the kind of the polymeric material of the 3-dimensional scaffold 401.

The 3-dimensional scaffold is provided to three-dimensionally culture cells therein or thereon, or to form a concentration gradient of a chemical thereacross through diffusion. Examples of the polymeric material for the 3-dimensional scaffold include Matrigel, Puramatrix, collagen, fibrin gel, PEGDA, and alginate.

Further, a mixture of various gels at a predetermined ratio may be also employed, and the ratio or the gels may be adjusted appropriately depending on the type of cells or the purpose of study.

For example, Matrigel may undergo sol-gel transition within several tens of minutes at room temperature or higher, to form a scaffold. For PEGDA, UV light or light around UV wavelengths elicits polymerization to form a scaffold. The injection of calcium ions can polymerize alginate into a scaffold.

In order to perform cell culturing within the 3-dimensional scaffold 401, cells may be mixed with the material for 3-dimensional scaffold before introduction to the second fluid path 201 while a culture medium for supplying nutrients to cells may be fed through the first fluid path 200 after the fabrication of the 3-dimensional scaffold 401.

For a cell culture on the surface of the 3-dimensional scaffold 401, cells may be preferably introduced via the first fluid path 200 after the fabrication of the 3-dimensional scaffold 401.

A concentration gradient of a chemical across the 3-dimensional scaffold 401 may be formed by injecting a solution of the chemical into the first fluid path 200 after the fabrication of the 3-dimensional scaffold 401. Once introduced into the first fluid path 200, the chemical migrates toward the 3-dimensional scaffold 401 by diffusion, and in the 3-dimensional scaffold 401, the concentration of the chemical is the highest at the side facing the solution of the chemical and gradually decreases with the distance from the side.

Accordingly, a concentration gradient can be formed across the 3-dimensional scaffold 401 and is dependent on various factors including diffusion properties, injection time, initial concentration of the chemical, and the type of the 3-dimensional scaffold. Also, the flow rate of the solution introduced may be a parameter of the concentration gradient.

In one embodiment of the present invention, the plate for forming the fluid paths 200 and 201 is made of PDMS while the substrate 101 is a polystyrene or glass substrate because these materials are inexpensive and easy to fabricate into the device.

In another embodiment, the second fluid path 201 in which the 3-dimensional scaffold 401 is filled is 100 μm high while the first fluid path 200 in which a culture medium is filled is 200 μm in height. The 3-dimensional scaffold 401 is 500 μm wide.

In another embodiment, the PDMS plate and the polystyrene substrate are attached to each other via treatment of the surfaces with silanes.

First, the polystyrene substrate is immersed in a 1% silane solution for 20 min at room temperature, and then washed.

Subsequently, the substrate is subjected to oxygen plasma for about 1 min, and overlaid with the PDMS plate which is also treated with oxygen plasma. In this manner, they can be readily attached to each other at room temperature.

Alternatively, a glass substrate may be employed. In this case, the glass substrate may be irreversibly attached to a PDMS plate only after treatment with oxygen plasma treatment even when it is not subjected to a special surface treatment. The more hydrophilic the surfaces of both of the plate 100 and the substrate are, the more advantageous it is in introducing and controlling fluid without the aid of a peripheral device such as a pump. Treatment with oxygen plasma makes surfaces of both the plate 100 and the substrate 101 hydrophilic. Since the contact angle of fluid with regard to the surfaces increases with time, fluids, such as a solution of a polymeric material for the 3-dimensional scaffold 401, a culture medium, etc. are preferably introduced within 1 hr after the attachment.

If two or more hours have passed, fluids may be introduced either with the aid of a peripheral device such as a pump, or after the surface treatment with oxygen plasma is conducted again.

Immersion in a silane solution maintains the surfaces in a hydrophilic state for several days to weeks, so that fluids can be readily introduced within the duration.

If there is a need of keeping for a longer time, immersion in water or alcohol rather than exposure to air prolongs the hydrophilicity of the surface.

In order to prevent the fluid to leaking to the first fluid path 200 after introduction to the second fluid path 201, the ratio of width to height of the second fluid path 201 is preferably maintained to be 4 or higher. Of course, even when the width/height ratio is smaller than this, only the second fluid path may be filled with fluid. If an excessive pressure is used, it causes the fluid to leak to the first fluid path 200 over the surface tension of fluid.

However, even when fluid leaks to the first fluid path 200, suction through the first inlet port 300 aspirates the fluid filled in the first fluid path 200 ahead of the fluid filled in the second fluid path 201. Thus, only the second fluid path 201 can readily be filled with fluid.

With reference to FIG. 5, there is a cross sectional view of the micro-device for culturing cells according to one embodiment of the present invention depicting the growth of cells within the 3-dimensional scaffold. FIGS. 6A and 6B are microscopic images of cells growing within the 3-dimensional scaffold of the micro-device for culturing cells.

In another embodiment, the cells 500 may be the breast cancer cell line MCF7 while the 3-dimensional scaffold 401 is a mixture of 1:1 of 10 mg/ml Matrigel and 2 mg/ml collagen type I. A total amount of 5 μL of the mixture is sufficient to form the scaffold.

In this embodiment, a 30% Matrigel-collagen solution mixed with cells was first introduced into the low-height fluid path, followed by solidifying the Matrigel-collagen solution for 30 min in a 37° C. incubator to form a 3-dimensional scaffold 401. Then, a culture medium is fed to the device through the high-height fluid paths 200 and 201.

At this time, a peripheral device such as a pump was not employed at all, and even when the fluid 400 is dropped to the inlet ports 300 and 301 by means of a pipette, it spontaneously fills the fluid paths 200 and 201 thanks to surface tension-driven capillary action. More hydrophilic surfaces of the fluid paths make the fluid filling the paths move faster.

Flowing from the low-height fluid path to the high-height fluid path is restrained by the surface tension of the fluid.

Conventionally, the volume of the 3-dimensional scaffold amounts to hundreds μL. In contrast, the microfluidic can not only remarkably reduce the volume of the 3-dimensional scaffold, but also decrease amounts of cells, media and reagents hundreds fold.

FIG. 7 is a cross sectional view of the micro-device for culturing cells of the present invention, depicting the generation of interstitial flow across the 3-dimensional scaffold.

In this embodiment, a second fluid path 201 with a low height in which a 3-dimentional scaffold is formed is disposed between two high-height, first fluid paths 200.

One of the first fluid paths 200 is named “1-1 fluid path” while the other is named “1-2 fluid path.”

The 1-1 fluid path and the 1-2 fluid path are separated from each other by the 3-dimensional scaffold 401 located in the second fluid path 201. Since the 3-dimensional scaffold 401 has pores with a size of several micrometers, the fluid can move in the direction indicated by arrows. This movement is called interstitial flow.

Generally, when the fluid pressure in the 1-1 fluid path is identical to that in the 1-2 fluid path, the 3-dimensional scaffold 401 allows only the diffusion of a chemical present in the fluid. If the fluid pressure differs from the 1-1 fluid path to the 1-2 fluid path, interstitial flow may occur.

A difference in internal fluid pressure between the 1-1 fluid path and the 1-2 fluid path may incur a difference in the height of fluid between respective inlet ports for the 1-1 fluid path and the 1-2 fluid path. In this context, the pressure difference can be represented by a product of the density of fluid, the gravitional acceleration, and the height difference of fluid. The rate of the interstitial flow increases with an increase in the pressure difference. In addition, the interstitial flow becomes faster as the fluid conductivity of the 3-dimensional scaffold 401 increases in conductivity or as the 3-dimensional scaffold 401 narrows.

The fluid conductivity of the 3-dimensional scaffold 401 varies depending on the material of the 3-dimensional scaffold 401. For example, Matrigel comprises 60% of collagen IV, 33% of laminin, and 5.4% of heparin sulfate the fibers of which have diameters of 0.7 nm, 0.6 nm, and 0.5 nm, respectively. In 20 mg/ml Matrigel, thus, an average distance from one fiber surface to another is on the order of 8 nm while 300 mg/ml Matrigel has an average inter-fiber distance of about 0.54 nm. That is, the average distance between the fibers decreases with an increase in the concentration of Matrigel, resulting in a decrease in the fluid conductivity of the 3-dimensional scaffold which, in turn, makes the rate of the interstitial flow slow down.

Accordingly, the cells can be controlled in terms of growth rate, differentiation and migration by modulating the rate of the interstitial flow or the concentration gradient of the chemical across the 3-dimentional scaffold or by mechanically stimulating the cells 500 cultured within the 3-dimensional scaffold. For instance, cell migration can be observed when a concentration gradient of a chemotactic compound is given in the scaffold.

FIG. 8A illustrates an embodiment in which a concentration gradient of a compound of interest is established across the 3-dimensional scaffold by introducing a solution containing the chemical to one of the fluid paths and a solution free of the chemical to the other. FIG. 8B depicts concentration gradients of a chemical of interest established across the 3-dimensional scaffold when the chemical is introduced to only one of the fluid paths as illustrated in FIG. 8A. In FIG. 8B, a concentration gradient of a chemical of interest is established by diffusion based on the concentration difference of the molecule, and the chemical tends to migrate from a high-concentration locus to a lower concentration one. In addition, not only does the steady introduction of the chemical to the fluid path allow the concentration gradient of the chemical to be kept constant, but the concentration gradient can be arbitrarily controlled by adjusting the rate of the interstitial flow.

A modulation in the rate of fluid flow or the pressure of the fluid within each of the fluid paths may have an influence on the interstitial flow or the concentration gradient of the chemical of interest.

For optical identification of the concentration gradient of a molecule across the 3-dimensional scaffold, FITC-labeled dextran of 10 KDa may be employed in one embodiment of the present invention.

FIG. 9 depicts cell migration within the 3-dimensional scaffold according to the concentration gradient. In FIG. 9, the 3-dimensional scaffold in which cells are cultured is disposed between two separated fluid paths which are positioned up- and downstream of the scaffold. A chemotactic agent which induces cell migration is introduced in mixture of a culture medium to the lower fluid path while a typical culture medium is fed into the upper fluid path. Under the condition, a concentration gradient is established across the 3-dimensional scaffold, demonstrating the cell migration driven by the chemotaxis.

In one embodiment of the present invention, the macrophage line RAW 264.7 is employed while a concentration gradient of CCL2, a chemokine, was established to induce the chemotactic migration of the macrophage cells.

In the cell culture device of the present invention, different types of cells may be cultured simultaneously. Actually, various cells are present in the body where a group of cells are located at a distance from another group, interacting with each other. To observe growth, migration and differentiation of cells in an environment mimic to the body is very important for conducting accurate tests in pharmacology, cell biology and molecular biology studies.

FIG. 10 depicts a micro-device for culturing cells according to another embodiment of the present invention in which heterogeneous cells are cultured simultaneously.

In this case, at least three fluid paths 200, 201 and 202 different in height from one another are provided, with a connection thereamong. In the fluid path 201 with the lowest height, a 3-dimensional scaffold 401 comprising one type of cells 500 is established while another 3-dimensional scaffold 402 containing another type of cells is introduced to the fluid path 202 which is the second lowest in height. Finally, a fluid 400 containing a culture medium or a stimulant chemical is fed to the fluid path 200 to culture the heterogeneous cells, together.

FIG. 11 is a photograph depicting the co-culturing of heterogeneous cells according to another embodiment of the present invention.

In this embodiment, the macrophage cell line RAW 264.7 and the breast cancer cell line MDA-MB-231 are co-cultured to observe the movement of the immune cells in a cancer microenvironment. A material for the 3-dimensional scaffold is a Matrigel/collagen hydrogel.

Macrophages 500 labeled with a fluorescent are mixed with the hydrogel 401 and introduced to the lowest-height fluid path 201, followed by incubation at 37° C. for about 30 min to solidify the gel. Subsequently, the breast cancer cells 501 dyed with a different fluorescent are mixed with the hydrogel 402, introduced to the fluid path 202 communicating with the lowest-height fluid path, and then incubated for about 30 min to establish a scaffold. Finally, a medium useful for co-culturing the two different types of cells is fed to the other fluid path 200. Upon the solidification of the gel, the micro-device for culturing cells is placed upside down to prevent the cells from settling down, thereby maintaining the cells in a 3-dimensional pattern. The two different types of cells are effectively fixed within the 3-dimensional scaffolds formed in the fluid paths. Since all the fluid paths are connected with one another, the culture medium and the signaling chemical can be delivered to the cells located in different sites.

FIG. 12 depicts a micro-device for culturing cells according to another embodiment of the present invention in which heterogeneous cells are cultured simultaneously.

As can be seen, plural fluid paths 200 and 201 with two different heights are constructed to provide five compartments in which two or more different types of cells are co-cultured. The plural fluid paths have respective inlet ports so that fluids can be selectively introduced to the fluid paths via the respective inlet ports.

First, a material for a 3-dimensional scaffold 401 containing one type of cells 500 is introduced to a fluid path 201 relatively low in height, and solidified. On the other hand, a fluid path 203 with a relatively high height is filled with a material for a 3-dimensional scaffold 402 containing another type of cells 501, followed by solidifying the material to form the scaffold. Then, a fluid 400 containing a culture medium or a stimulant chemical is introduced to a fluid path 200 to culture the heterogeneous cells simultaneously.

FIG. 13 is a photograph illustrating an embodiment in which heterogeneous cells are co-cultured.

In this embodiment, the breast cancer cell line MDA-MB-231, and fibroblasts are dyed with respective fluorescents. For the 3-dimensional scaffold, Matrigel/collagen hydrogel is employed.

First, fluorescent-labeled fibroblasts 500 in mixture with the hydrogel 401 are introduced to a relatively low-height fluid path 201, and incubated at 37° C. for about 30 min to solidify the gel. Then, the breast cancer cell line 501 labeled with a different fluorescent is mixed with the hydrogel 402 and introduced to a relatively high-height fluid path 203 which is located between the relatively low-height fluid paths 201, followed by solidification for about 30 min. Finally, a medium for co-culturing the two different types of cells is fed to the other fluid path 200. Upon the solidification of the gel, the micro-device for culturing cells is placed upside down to prevent the cells from settling down, thereby maintaining the cells in a 3-dimensional pattern. The two different types of cells are effectively fixed within the 3-dimensional scaffolds formed in the fluid paths. Since all the fluid paths are connected with one another, the culture medium and the signaling chemical can be delivered to the cells located in different sites.

In this manner, as shown in FIG. 12, two or more different types of cells may be co-cultured in respective fluid paths.

Further, two or more types of stromal cells, such as fibroblasts, may be cultured in a mixture in one compartment. That is, different combinations of cells may be introduced in mixture with a scaffold material to respective fluid paths to construct versatile types of multi-cell co-culture platforms.

In the micro-device for culturing cells of the present invention, as described above, when fluid is introduced to the relatively low-height fluid path, this path is first filled with the fluid due to surface tension. However, fluid may be first introduced to the relatively high-height fluid path.

FIG. 14 is a cross sectional view illustrating an embodiment in which fluid is introduced to a relatively high-height fluid path.

The micro-device for culturing cells of FIG. 14 may comprise at least two fluid paths 700 and 702 through which fluid moves; a fluid path 701, positioned between the at least two fluid paths 700 and 702, having a height lower than that of the at least two fluid paths 700 and 702; and an inlet port (not shown) for injecting fluid to fluid paths 700 and 702.

In this structure, the flow of fluid from the fluid path 701 to the fluid paths 700 and 702 is restrained by the surface tension of the fluid introduced, and fluid first fills the fluid paths 700 and 702 to which the fluid is introduced, and the fluid path 701.

When employing a 3-dimensional scaffold, the micro-device for culturing cells may comprise at least two fluid paths 700 and 702 through which fluid move; a 3-dimensional scaffold 401 in contact with the fluid paths 700 and 702; a fluid path 701, positioned between the fluid paths 700 and 702, being higher in height than the fluid paths 700 and 702 and the 3-dimensional scaffold; and inlet ports (not shown) for introducing fluid to fluid paths 700 and 702.

FIG. 15 is a process flow depicting the construction of the microfluidic cell culture according to the present invention.

As can be seen in FIG. 15, first, a microstructure 801 having the same height as a first fluid path is formed in an area of a flat plate 800, said area corresponding to a sum of the first fluid path and a second fluid path lower in height than the first fluid path (S100).

Next, a microstructure 803 with a height corresponding to a difference between the heights of the first fluid path and the second fluid path is formed on the microstructure 801 (S200).

Subsequently, a liquid material 100 is applied to the imposed microstructures 801 and 803, and solidified (S300).

Then, the solidified material 100 is overlaid to a substrate 101 to form fluid paths 200 and 201 (S400).

A polymeric material for a 3-dimensional scaffold is introduced to the second fluid path (S500).

Finally, fluid is injected to the first fluid path (S600).

In steps S100 and S200, various methods may be taken to form the microstructures 801 and 803.

When photolithography is to be employed, for example, a photoresist is applied to a certain area, followed by UV irradiation to form microstructures after the area.

Alternatively, tapes are cut out and imposed to each other, as shown in FIGS. 15 and 16. This is a very cheap method for fabricating the microstructures.

In FIG. 16, a tape sheet after clipping is shown in the right panel while clipped tapes for fabricating microstructures are arranged in the left panel.

After being poured to the microstructures, a molding material is solidified, and the mold thus obtained is detached, and junctioned to the substrate to construct fluid paths.

In another embodiment of the present invention, PDMS is used as the molding material for fluid paths, and mixed at a ratio of 10:1 with a curing agent before application. This mixture is poured, and then solidified at 65° C. for 2 hrs.

Thereafter, the PDMS mold is detached, treated with oxygen plasma, together with, for example, a glass substrate, and junctioned to the glass substrate. Finally, a fluid containing a 3-dimensional scaffold material is introduced to the fluid path to fabricate a micro-device for culturing cells according to the present invention.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A micro-device for culturing cells, comprising:
a plurality of fluid paths through which fluid moves; and
at least one inlet port for injecting fluid to the fluid paths, said fluid paths communicating with each other and being different in height from each other.

2. The micro-device for culturing cells of claim 1, wherein when fluid is injected via the inlet port, a fluid path with a relatively lower height is preferentially filled with the fluid.

3. The micro-device for culturing cells of claim 1, wherein at least one of the fluid paths contains a 3-dimensional scaffold, formed of a polymeric material, for culturing cells therein.

4. The micro-device for culturing cells of claim 3, further comprising a design for inducing the polymeric material to solidify, said design being associated with a temperature, a time, a light wavelength, or an agent.

5. The micro-device for culturing cells of claim 3, wherein the polymeric material is selected from the group consisting of Matrigel, Puramatrix, collagen, fibrin gel, PEGDA, alginate, and a combination thereof.

6. The micro-device for culturing cells of claim 3, wherein a culture medium is fed to at least one fluid path different from the fluid path containing the 3-dimensional scaffold to provide a nutrient for cells growing in the 3-dimensional scaffold, and the 3-dimensional scaffold is established using a polymeric material in mixture with cells.

7. The micro-device for culturing cells of claim 3, wherein the fluid path containing the 3-dimensional scaffold is provided with cells by injection thereto so that the cells are allowed to grow on the surface of the 3-dimensional scaffold.

8. The micro-device for culturing cells of claim 3, wherein a chemical is introduced to a fluid path different from the fluid path containing the 3-dimensional scaffold to establish a concentration gradient of the chemical across the 3-dimensional scaffold, said concentration gradient varying depending on diffusion properties, sizes, injection times, and initial concentration differences of the chemical, kind of the 3-dimensional scaffold, and rates of injected solution.

9. The micro-device for culturing cells of claim 3, wherein the 3-dimensional scaffold is established within two or more fluid paths, different in height from each other, in which different types or combinations of cells are respectively cultured, whereby heterogeneous cells are co-cultured therein.

10. The micro-device for culturing cells of claim 1, wherein the fluid paths are formed of a material selected from the group consisting of poly(dimethylsiloxane) (PDMS), polymethylmethacrylate (PMMA), polyacrylates, polycarbonates, polycyclic olefins, polyimides, polyurethanes, polystyrene, glass, and a combination thereof.

11. A method for manufacturing micro-device for culturing cells, comprising:
forming a first microstructure having the same height as a first fluid path on an area of a flat plate, said area corresponding to a sum of the first fluid path and a second fluid path lower in height than the first fluid path;
forming a second microstructure having a height corresponding to a difference between the heights of the first fluid path and the second fluid path on the first microstructure;
applying a liquid material for forming the fluid paths to the first microstructure and the second microstructure, followed by solidifying the liquid material;
overlying the solidified material to a substrate to form the first fluid path and the second fluid path, with a connection therebetween;
introducing a polymeric material to the second fluid path to form a 3-dimensional scaffold; and
injecting fluid to the first fluid path.

12. The method of claim 11, wherein the liquid material for forming the fluid paths is selected from the group consisting of poly(dimethylsiloxane) (PDMS), polymethylmethacrylate (PMMA), polyacrylates, polycarbonates, polycyclic olefins, polyimides, polyurethanes, polystyrene, glass, and a combination thereof.

13. The method of claim 11, wherein the fluid path has a hydrophilic surface, and the fluid is injected at a contact angle of 90° or less to the first fluid path.

14. The method of claim 11, wherein the polymeric material is selected from the group consisting of Matrigel, Puramatrix, collagen, fibrin gel, PEGDA, alginate, and a combination thereof.

15. The method of claim 11, further providing a factor for inducing the polymeric material to solidify, said factor being selected from the group consisting of a temperature, a time, a light wavelength, and an agent.

16. The method of claim 11, wherein the first and the second microstructures are formed using a cut-out tape or using a process selected from among photolithography, an imprinting process and a hot embossing process.

17. The method of claim 11, wherein the second fluid path is composed of two or more fluid paths which are different in height from each other, with a connection therebetween, and the 3-dimensional scaffold is sequentially formed within the fluid paths from lower to higher heights.

18. A method for culturing cells using the micro-device for culturing cells having a plurality of fluid paths, comprising:
introducing a polymeric material to at least one fluid path having a relatively low height;
solidifying the polymeric material to form a 3-dimensional scaffold; and
injecting fluid for cell culture to a fluid path in contact with the 3-dimensional scaffold.

19. The method of claim 18, wherein a culture medium is fed to at least one fluid path different from the fluid path containing the 3-dimensional scaffold to provide a nutrient for cells growing in the 3-dimensional scaffold, and the 3-dimensional scaffold is established using a polymeric material in mixture with cells.

20. The method of claim 18, wherein the fluid path containing the 3-dimensional scaffold is provided with cells by injection thereto so that the cells are allowed to grow on the surface of the 3-dimensional scaffold.

21. The method of claim 18, wherein a chemical is introduced to a fluid path different from the fluid path containing the 3-dimensional scaffold to establish a concentration gradient of the chemical across the 3-dimensional scaffold, said concentration gradient varying depending on diffusion properties, sizes, injection times, and initial concentration differences of the chemical, kind of the 3-dimensional scaffold, and rates of injected solution.
